(19) United States
(12) Patent Application Publication
(10) Pub. No.: US 2004/0138216 A1
(43) Pub. Date: Jul. 15, 2004

(54) Process for the preparation of an essentially pure polymorph of an n-pyrazolyl-n'-naphthyl-urea

(75) Inventors: Wendelin Samstag, Bad Kreuznach (DE); Gunter Koch, Schwabenheim (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/727,214

(22) Filed: Dec. 3, 2003

Related U.S. Application Data
(60) Provisional application No. 60/436,136, filed on Dec. 23, 2002.

(51) Int. Cl. A61K 31/5377; C07D 413/02

(52) U.S. Cl. 514/235.2; 544/140

(57) ABSTRACT

Disclosed are improved processes for the preparation of a polymorph of 1-[tert-butyl-1-p-tolyl-1H-pyrazol-5-yl]-3-[4-(2-morpolinin-4-yl-ethoxy)naphthalen-1-yl]-urea (1) by crystallization from an alcohol, wherein the improvement is that crude (1) is treated with ethanol.

### APPLICATION DATA

This application claims benefit to U.S. provisional application No. 60/436,136 filed Dec. 23, 2002.

### BACKGROUND OF THE INVENTION

1. Technical Field

The invention relates to an improved process for the preparation of a polymorph of 1-[tert-butyl-1-p-tolyl-1H-pyrazol-5-yl]-3-[4-(2-morpolinin4-yl-ethoxy)naphthalen-1-yl]-urea (1) by crystallization from an alcohol.

2. Background Information

1-[tert-butyl-1-p-tolyl-1H-pyrazol-5-yl]-3-[4-(2-morpolinin-4-yl-ethoxy)naphthalen-1-yl]-urea (1) is an therapeutically effective inhibitor of cytokines, which can be used for treating diseases and pathological conditions involving inflammations. This compound is known for example from the US patent U.S. Pat. No. 6,319,921.

The International patent application WO 01/04115 suggests a process for the preparation of (1) by a condensation reaction between 4-amino-1-(2-morpholinoethoxy)naphthalene and 5-(2,2,2-trichloroethoxycarbonyl)amino-3-tert-butyl-1-p-tolylpyrazole. The resulting crude product is crystallized from acetonitrile and re-crystallized from isopropanol and yields a polymorph Form 1 which is contaminated by other polymorphs (Form 2, Form 3, . . . ) and undesired by-products.

Accordingly, there is a high need to provide the polymorph of (1) in an essentially pure form.

### BRIEF SUMMARY OF THE INVENTION

Surprisingly, it has been found that the essentially pure polymorph of (1) can be obtained, when crude (1) is treated with ethanol.

Accordingly, the invention relates to an improved process for the preparation of a polymorph of 1-[tert-butyl-1-p-tolyl-1H-pyrazol-5-yl]-3-[4-(2-morpolinin-4-yl-ethoxy)naphthalen-1-yl]-urea (1) by crystallization from an alcohol, wherein the improvement is that crude (1) is treated with ethanol.

Another aspect of the invention is the essentially pure polymorph of 1-[tert-butyl-1-p-tolyl-1H-pyrazol-5-yl]-3-[4-(2-morpolinin-4-yl-ethoxy)naphthalen-1-yl]-urea (1) which is obtainable by crystallization of the crude (1) with ethanol.

Furthermore, the invention relates to a pharmaceutical composition comprising an effective amount of the essentially pure polymorph Form 1 of (1) in combination with at least one pharmaceutical excipient and to a method of treating an inflammatory disease which comprises administering to a patient in need of such treatment a therapeutically effective amount of an essentially pure polymorph of (1).

### DETAILED DESCRIPTION OF THE INVENTION

It has now been found that if (1) is crystallized from a solvent like acetonitril and/or isopropanol, the crystalline form, Form 1, corresponding to that of the product obtained according to WO 01/04115 mentioned above is, but contains different by-products and other polymorphs such as Forms 2 and 3.

Furthermore, Form 1, which is not contaminated by said by-products and other polymorphs is more compact and much less electrostatic than the contaminated Form 1 and may hence be more readily subjected to any treatment under the usual conditions of pharmaceutical technology and, in particular, of formulation on an industrial scale.

The term “essentially pure” as used hereinbefore and hereinbelow relates to the polymorph Form 1, which is essentially free of other polymorphic forms and by-products. As a rule it consists of at least 98%, preferably of 98.5 to 100%, in particular of 99.85 to 99.99% of said Form 1.

The term “treating with ethanol” as used hereinabove and hereinbelow encompasses any procedural step, in which crude (1) is brought in to contact with ethanol, preferably adding ethanol to (1) at ambient or elevated temperature, dissolving crude (1) in ethanol, optionally in the presence of a polar aprotic co-solvent such as for example DMSO, crystallizing (1) from a solution of (1) in ethanol by seeding with crystals of (1), by lowering the temperature and/or by dilution with water. Furthermore, the term “treating with ethanol” includes washing of solid product (1) with ethanol, optionally followed by subsequent washing with water.

In a preferred embodiment crude (1) is first re-crystallized from ethanol and the solid product obtained by re-crystallization is subsequently washed with ethanol and water.

The term “ethanol” as used hereinabove and hereinbelow encompasses technically pure ethanol having a purity of 95% which is essentially free of water.

Preferably the crude (1) is treated with ethanol at a temperature from 0° C. to 80° C., in particular from 10 to 60° C., most preferably from 25 to 50° C.

Furthermore preferred is a process, wherein 1 part per weight of crude (1) is treated with 2 to 50, in particular 3 to 20 parts per weight ethanol with respect to (1).

In a particularly preferred embodiment the process according to the invention comprises the steps of

(a) dissolving crude (1) with ethanol, in particular in admixture with a polar aprotic solvent such as DMSO, acetonitrile, dimethylformamide or ethyl acetate, most preferably DMSO,

(b) adding seeding crystals of the pure polymorph of (1),

(c) allowing the pure polymorph of (1) to crystallize, preferably by allowing the crystals formed to settle by interrupted stirring,

(d) adding water until the crystallization is almost completed,

(e) separation of the pure polymorph of (1), preferably by filtration, sedimentation, decantation and/or centrifugation, and

(f) optionally washing the resulting pure polymorph of (1) with ethanol and/or water and drying at elevated temperature.

Most preferably a mixture of 1 part per weight of crude (1) obtained according to the method described in example 1 of WO 01/04115, DMSO (0.5 to 2.5, in particular 1.5 part per weight) and ethanol (0.1 to 2.0, in particular 1.0 part per weight) is stirred at 0° C. to 45° C., in particular at ambient temperature for 1 to 60, in particular 35 min. Charcoal (0.01 to 0.5, in particular 0.1 part per weight) is added to the resulting solution. The suspension is filtered and the residue washed with ethanol (1 to 5, in particular 3 part per weight). Seeding crystals are added to the resulting solution; subsequently water (2 to 10, in particular 4 part per weight) is added at 0° C. to 45° C., in particular at 20-30° C. The resulting suspension is cooled to ambient temperature and the solid product is isolated and washed with ethanol and water. The resulting crystals are dried at elevated temperature in particular at 40 to 70° C. to yield white crystals of polymorph Form 1 of (1) with a purity of >99.5%

In another preferred embodiment of the present 1,01-1,1 equivalents of 4-amino-1-(2-morpholinoethoxy)naphthalene (2) are reacted with 1 equivalent of 5-(2,2,2-trichloroethoxycarbonyl)amino-3-tert-butyl-1-p-tolylpyrazole (3) in the presence of 1 equivalent of a tertiary amine and a solvent consisting of DMSO, preferably 0.5 to 1.5, in particular about 0.8 to 1.1 parts per weight of DMSO with respect to (2) and ethyl acetate, preferably 1.0 to 10.0, in particular about 6.0 to 8.0 parts per weight of ethyl acetate with respect to (2); isolation of crude (1); washing crude (1) with ethyl acetate; and treating the residue with ethanol.

Preferred tertiary amines for example are triethylamine, diisopropylethylamine, N-methylpyrrolidine, DBU(1,8-diazabicyclo[5.4.0]undec-7-ene), DMAP(4-dimethylaminopyridine), N-methylmorpholine, pyridine or methyl pyridine. Most preferred tertiary amines are diisopropylethylamine or N-methylpyrrolidine. The reaction occurs at a temperature of about 0-100° C., preferably 5-15° C., for about 0.5-24 hrs, preferably 3-4 hrs.

In a particularly preferred embodiment a solution consisting essentially of 5-(2,2,2-trichloroethoxycarbonyl)amino-3-t-butyl-1-p-tolylpyrazole (3) (1 equivalent), 4-amino-1-(2-morpholinethoxy)naphthalene (2) (free base, 1.02-1.08 equivalents), diisopropylethylamine (1 equivalent), DMSO (0.5 to 1.5 parts per weight with respect to (2)) and ethyl acetate (1.0 to 10.0 parts per weight with respect to (2)) is stirred at 60-100° C. The mixture was allowed to cool to ambient temperature and stirred for 16 hrs at ambient temperature. Charcoal is added to the resulting solution. The resulting suspension is filtered and the residue washed with ethyl acetate. The organic layer is concentrated in vacuo. The residue is treated with ethanol and seeding crystals and subsequently water are added. The resulting solid product is isolated and washed with ethanol and water. The resulting crystals are dried at to yield white crystals of polymorph Form 1 of (1) with a purity of at least 99.5%.

The pure polymorph of (1) is characterized by the following X-ray powder diffractogram (XRPD), which is analyzed using an X-Ray Powder Diffractometer utilizing CuKα radiation (γ=1.5418 Å), run at 40 kV, 30 mA:

| Peak Position (° 2θ) | Relative Intensity | d-Space (Å) |
|---|---|---|
| 5.4 | 38 | 16.4 |
| 7.0 | 14 | 12.7 |
| 8.9 | 46 | 9.90 |
| 10.4 | 66 | 8.54 |
| 10.7 | 7 | 8.31 |
| 11.5 | 17 | 7.68 |
| 12.6 | 7 | 7.05 |
| 13.8 | 50 | 6.41 |
| 14.3 | 100 | 6.18 |
| 15.5 | 7 | 5.71 |
| 15.9 | 9 | 5.58 |
| 16.5 | 17 | 5.38 |
| 17.1 | 75 | 5.19 |
| 18.2 | 19 | 4.87 |
| 18.7 | 19 | 4.76 |
| 19.1 | 39 | 4.65 |
| 20.0 | 32 | 4.45 |
| 20.7 | 79 | 4.29 |
| 21.0 | 45 | 4.24 |
| 21.7 | 35 | 4.09 |
| 22.8 | 47 | 3.90 |
| 23.3 | 28 | 3.82 |
| 23.8 | 33 | 3.73 |
| 24.7 | 26 | 3.60 |
| 25.1 | 26 | 3.55 |
| 25.8 | 22 | 3.45 |
| 26.3 | 21 | 3.39 |
| 26.4 | 21 | 3.37 |
| 26.6 | 20 | 3.35 |
| 26.8 | 16 | 3.33 |
| 28.0 | 15 | 3.19 |
| 28.4 | 16 | 3.14 |
| 29.8 | 9 | 2.99 |
| 31.6 | 9 | 2.83 |
| 32.9 | 16 | 2.72 |
| 34.0 | 12 | 2.64 |

Surprisingly the process according to the invention allows to manufacture the pure polymorph of (1) in higher purity and yields. Moreover, the process according to the invention for the preparation of (1) from (2) and (3) can be carried out with lower amounts of solvents than suggested by WO 01/04115, which is important for production in industrial scale with respect to environmental requirements and solvent management.

The essentially pure polymorph of the invention is useful for the treatment of inflammatory conditions. These encompass chronic inflammatory diseases including, but not limited to, osteoarthritis, multiple sclerosis, Guillain-Barre syndrome, Crohn's disease, ulcerative colitis, psoriasis, graft versus host disease, systemic lupus erythematosus and insulin-dependent diabetes mellitus. The essentially pure polymorph of the invention can also be used to treat other disorders associated with the activity of elevated levels of proinflammatory cytokines such as responses to various infectious agents and a number of diseases of autoimmunity such as rheumatoid arthritis, toxic shock syndrome, diabetes and inflammatory bowel diseases.

In addition, the essentially pure polymorph of the invention being an inhibitor of cytokine production are expected to block inducible cyclooxygenase (COX-2) expression. COX-2 expression has been shown to be increased by cytokines and it is believed to be the isoform of cyclooxygenase responsible for inflammation (M. K. O'Banion et al., Proc. Natl. Acad. Sci. U.S.A., 1992, 89, 4888.) Accordingly, the essentially pure polymorph would be expected to exhibit efficacy against those disorders currently treated with COX inhibitors such as the familiar NSAIDs. These disorders include acute and chronic pain as well as symptoms of inflammation and cardiovascular disease.

Furthermore, the essentially pure polymorph of the invention may be useful in the treatment of diseases mediated predominantly by neutrophils such as stroke and myocardial infarction, alone or following thrombolytic therapy, thermal injury, adult respiratory distress syndrome (ARDS), multiple organ injury secondary to trauma, acute glomerulonephritis, dermatoses with acute inflammatory components, acute purulent meningitis or other central nervous system disorders, hemodialysis, leukopherisis, granulocyte transfusion associated syndromes, and necrotizing entrerocolitis.

For therapeutic use, the essentially pure polymorph of the invention may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The essentially pure polymorph of this invention may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. The essentially pure polymorph of the invention may be physically combined with the conventional therapeutics or other adjuvants into a single pharmaceutical composition. Advantageously, The essentially pure polymorph may then be administered together in a single dosage form. In some embodiments, the pharmaceutical compositions comprising such combinations of compounds contain at least about 5%, but more preferably at least about 20%, of an essentially pure polymorph (w/w) or a combination thereof. The optimum percentage (w/w) of an essentially pure polymorph may vary and is within the purview of those skilled in the art. Alternatively, the essentially pure polymorph may be administered separately (either serially or in parallel). Separate dosing allows for greater flexibility in the dosing regime.

As mentioned above, dosage forms of the essentially pure polymorph of this invention include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 10-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

Procedures by way of example for preparing the essentially pure polymorph according to the invention will be described in more detail hereinafter. The Examples which follow serve solely as a detailed illustration without restricting the subject matter of the invention.

### EXAMPLE 1

### 1-[3-tert-butyl-1-p-tolyl-1H-pyrazol-5-yl]-3-[4-(2-morpholin-4-yl-ethoxy)naphthalen-1-yl]-urea (1)

A solution of 5-(2,2,2-trichloroethoxycarbonyl)amino-3-t-butyl-1-p-tolylpyrazole (29.7 g, 73.4 mmol), 4-amino-1-(2-morpholinethoxy)naphthalene (free base, 20.4 g, 74.9 mmol), diisopropylethylamine (9.5 g, 73.4 mmol), DMSO (20 mL) and ethyl acetate (10 mL) was heated to 80-85° C. and stirred for 4.5 hrs. The mixture was allowed to cool to ambient temperature and stirred for 16 hrs at ambient temperature. To this solution, charcoal (2.0 g) was added. The suspension was filtered and the residue washed with ethyl acetate (70 mL) The organic layer was concentrated in vacuo (at 50-60° C. and 50-200 mbar). The residue is treated with ethanol at 35-40° C. Seeding crystals were added to the clear solution; subsequently water (150 ml) was added. The resulting suspension is cooled to ambient temperature and the solid product was isolated and washed with ethanol and water. The resulting crystals are dried at 60° C. to yield 35.13 g (90.8%) of white crystals of polymorph Form 1 of (1) with a purity of 99.9%.

### EXAMPLE 2

### 1-[3-tert-butyl-1-p-tolyl-1H-pyrazol-5-yl]-3-[4-(2-morpholin-4-yl-ethoxy)naphthalen-1-yl]-urea (1)

A mixture of crude (1) (30.0 g, 56.9 mmol) obtained according to example 1 of WO 01/04115, DMSO (45.0 ml) and ethanol (30 ml) was stirred at ambient temperature for 35 min. Charcoal (3.0 g) was added to the resulting solution. The suspension was filtered and the residue washed with ethanol (105 ml) Seeding crystals were added to the clear solution; subsequently water (120 ml) was added at 20-30° C. The resulting suspension is cooled to ambient temperature and the solid product was isolated and washed with ethanol and water. The resulting crystals are dried at 50° C. to yield 29.1 g (97.0%) of white crystals of polymorph Form 1 of (1) with a purity of 99.91%.

What is claimed is:

1. A process for the preparation of a polymorph of 1-[tert-butyl-1-p-tolyl-1H-pyrazol-5-yl]-3-[4-(2-morpolinin-4-yl-ethoxy)naphthalen-1-yl]-urea (1) by crystallization from an alcohol, said process comprising treating a crude 1-[tert-butyl-1-p-tolyl-1H-pyrazol-5-yl]-3-[4-(2-morpolinin-4-yl-ethoxy)naphthalen-1-yl]-urea (1) with ethanol.

2. The process according to claim 1, wherein crude (1) is treated with ethanol at a temperature from 0° C. to 80° C.

3. The process according to claim 2, wherein 1 part per weight of crude (1) is treated with 2 to 50 parts per weight ethanol.

4. A process for the preparation of a polymorph of 1-[tert-butyl-1-p-tolyl-1H-pyrazol-5-yl]-3-[4-(2-morpolinin-4-yl-ethoxy)naphthalen-1-yl]-urea (1) by crystallization from an alcohol, said process comprising:
(a) dissolving crude (1) with ethanol,
(b) adding seeding crystals of the pure polymorph of (1),
(c) allowing the pure polymorph of (1) to crystallize,
(d) adding water until the crystallization is almost completed,
(e) separating of the pure polymorph of (1), and
(f) optionally washing the resulting pure polymorph of (1) with water and drying at elevated temperature and/or in vacuo.

5. A process for the preparation of a polymorph of 1-[tert-butyl-1-p-tolyl-1H-pyrazol-5-yl]-3-[4-(2-morpolinin-4-yl-ethoxy)naphthalen-1-yl]-urea (1) by crystallization from an alcohol, said process comprising:
(i) treating 1.01-1.1 mole of 4-amino-1-(2-morpholinoethoxy)naphthalene (2) with 1 mole of 5-(2,2,2-trichloroethoxycarbonyl)amino-3-tert-butyl-1-p-tolylpyrazole (3) in the presence of 1 mole of a secondary amine and a solvent consisting of DMSO and ethyl acetate to produce crude (1);
(ii) isolating crude (1);
(iii) washing crude (1) with ethyl acetate and
(iv) treating the residue with ethanol.

6. The process according to any one of claims 1, 4 or 5, wherein the pure polymorph of (1) is characterized by the following X-ray powder diffractogramm (XRPD), which is analyzed using an X-Ray Powder Diffractometer utilizing CuKα radiation (γ=1.5418 Å), run at 40 kV, 30 mA:

| Peak Position (° 2θ) | Relative Intensity | d-Space (Å) |
|---|---|---|
| 5.4 | 38 | 16.4 |
| 7.0 | 14 | 12.7 |
| 8.9 | 46 | 9.90 |
| 10.4 | 66 | 8.54 |
| 10.7 | 7 | 8.31 |
| 11.5 | 17 | 7.68 |
| 12.6 | 7 | 7.05 |
| 13.8 | 50 | 6.41 |
| 14.3 | 100 | 6.18 |
| 15.5 | 7 | 5.71 |
| 15.9 | 9 | 5.58 |
| 16.5 | 17 | 5.38 |
| 17.1 | 75 | 5.19 |
| 18.2 | 19 | 4.87 |
| 18.7 | 19 | 4.76 |
| 19.1 | 39 | 4.65 |
| 20.0 | 32 | 4.45 |
| 20.7 | 79 | 4.29 |
| 21.0 | 45 | 4.24 |
| 21.7 | 35 | 4.09 |
| 22.8 | 47 | 3.90 |
| 23.3 | 28 | 3.82 |
| 23.8 | 33 | 3.73 |
| 24.7 | 26 | 3.60 |
| 25.1 | 26 | 3.55 |
| 25.8 | 22 | 3.45 |
| 26.3 | 21 | 3.39 |
| 26.4 | 21 | 3.37 |
| 26.6 | 20 | 3.35 |
| 26.8 | 16 | 3.33 |
| 28.0 | 15 | 3.19 |
| 28.4 | 16 | 3.14 |
| 29.8 | 9 | 2.99 |
| 31.6 | 9 | 2.83 |
| 32.9 | 16 | 2.72 |
| 34.0 | 12 | 2.64 |

7. An essentially pure product polymorph of 1-[tert-butyl-1-p-tolyl-1H-pyrazol-5-yl]-3-[4-(2-morpolinin-4-yl-ethoxy)naphthalen-1-yl]-urea (1) produced by the process according to claims 1, 4, 5 or 6.

8. A pharmaceutical composition comprising a pharmaceutically effective amount of the polymorph Form 1 of (1) according to claim 7 in combination with at least one pharmaceutical excipient.

9. A method of treating an inflammatory disease which comprises administering to a patient a therapeutically effective amount of a polymorph of (1) according to claim 7.
